(19) United States
(12) Patent Application Publication
(10) Pub. No.: US 2009/0292203 A1
(43) Pub. Date: Nov. 26, 2009

(54) ULTRASONIC IMAGING APPARATUS AND ULTRASONIC IMAGING SYSTEM

(76) Inventor: Shinichi Amemiya, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/469,529

(22) Filed: May 20, 2009

(30) Foreign Application Priority Data
May 23, 2008 (JP) 2008-135557

(51) Int. Cl. A61B 8/00; G06F 21/00

(52) U.S. Cl. 600/437; 726/31; 726/26

(57) ABSTRACT

An ultrasonic imaging apparatus includes a plurality of transmission/reception units each having a probe connector, a transmission/reception section, and a power supply, a plurality of display units each having an image display section and a power supply, a plurality of operation units each having an operation section and a power supply, a single control unit, and a communication means for communicably connecting said control unit to said transmission/reception units, said display units, and said operation units. The control unit has a unit control means for individually controlling said plurality of transmission/reception units, said plurality of display units, and said plurality of operation units, all of which are connected to said control unit, and a software management section for managing enable/disable of softwares for respectively controlling said plurality of display units and said plurality of operation units.

### CROSS REFERENCE TO RELATED APPLICATIONS

This application claims the benefit of Japanese Patent Application No. 2008-135557 filed May 23, 2008, which is hereby incorporated by reference in its entirety.

### BACKGROUND OF THE INVENTION

The embodiments described herein relate to an ultrasonic imaging system including functionally-distributed ultrasonic imaging apparatuses interconnected by a communication means, or including a plurality of ultrasonic imaging apparatuses interconnected by a communication means.

The increase in the use of ultrasonic imaging apparatus has been significant in recent years, and a large number of ultrasonic imaging apparatuses are often used in one hospital. Under such circumstances, reduction of costs for operating the ultrasonic imaging apparatuses is desirable from the view point of hospital management.

Further, along with functional sophistication of the ultrasonic imaging apparatus, a functionally-distributed ultrasonic imaging apparatus is also proposed (see, for example, Japanese Patent Application No. 2007-218282). In this apparatus, there are connected such components as a plurality of transmission/reception units for obtaining tomographic images by contacting an ultrasonic probe with a subject, a plurality of display units for displaying and interpreting the tomographic images, a plurality of operation units for inputting information of various controls, and a single control unit for forming topographic image information and processing various images, through a communication means. In this ultrasonic imaging apparatus, it is possible to manage and operate functions substantially equivalent to a plurality of ultrasonic imaging apparatuses, by controlling the plurality of transmission/reception units, the plurality of display units, and the plurality of operation units by means of the single control unit.

Further, with the adoption of LAN (Local Area Network) inside the hospital and the increasing use of high-speed communication networks outside the hospital, it becomes possible to easily exchange information, such as image data, among a plurality of ultrasonic imaging apparatuses placed inside and outside the hospital (see, for example, Japanese Unexamined Patent Publication No. 2000-259747).

However, according to the above described background art, software such as of an option function which is used in the functionally-distributed ultrasonic imaging apparatus and whose frequency of use is low, is not effectively used. In other words, in the functionally-distributed ultrasonic imaging apparatus described above, software such as of an option function is licensed for each of the plurality of transmission/reception units, the plurality of display units, or the plurality of operation units, according to the necessity.

In this state, a plurality of similar softwares are prepared in a single control unit. These softwares are not useless if constantly used. However, there occurs a case that software, such as of an option function whose frequency of use is low, constantly exists in an unused state. As a result, the use efficiency of the software is degraded.

### BRIEF DESCRIPTION OF THE INVENTION

An ultrasonic imaging apparatus according a first aspect of the invention includes: a plurality of transmission/reception units each having a probe connector, a transmission/reception section, and a power supply; a plurality of display units each having an image display section and a power supply; a plurality of operation units each having an operation section and a power supply; a single control unit; and a communication means for communicably connecting the transmission/reception units, the display units, and the operation units, to the control unit. The control unit has a unit control means for individually controlling the plurality of transmission/reception units, the plurality of display units, and the plurality of operation units, all of which are connected to the control unit, and has a software management section for managing enable/disable of softwares used in the unit control means to respectively control the plurality of transmission/reception units, the plurality of display units, and the plurality of operation units.

In the first aspect, the unit control means controls the transmission/reception units, the display units, and the operation units, all of which are connected to the unit control means, and the software management section integrally manages use or nonuse of software used in the transmission/reception unit, the display unit, and the operation unit.

As for the ultrasonic imaging apparatus according to a second aspect of the invention, in the ultrasonic imaging apparatus according to the first aspect, the software management section has a software determination means to determine, based on the selection information for selecting the software from at least one of the plurality of operation units through the unit control means, enable/disable of the software specified by the selection information.

In the second aspect, the softwares are enabled or disabled by the determination of the software determination means.

As for the ultrasonic imaging apparatus according to a third aspect of the invention, in the ultrasonic imaging apparatus according to the second aspect, the software is an option software which is not included as standard equipment in the ultrasonic imaging apparatus.

In the third aspect, the software whose frequency of use is low is used.

As for the ultrasonic imaging apparatus according to a fourth aspect of the invention, in the ultrasonic imaging apparatus according to the second or third aspect, the software determination means determines that when the software is offered from the source with licensing terms and conditions in which the maximum number of the software to be used is specified, the number of uses of the software in the unit control means is made equal to or less than the maximum number.

In the fourth aspect, the software determination means makes the number of the software to be used equal to or less than the maximum number that is the number of licenses.

As for the ultrasonic imaging apparatus according to a fifth aspect of the invention, in the ultrasonic imaging apparatus according to the fourth aspect, the use includes copying for only making copies, without involving execution of the software.

As for the ultrasonic imaging apparatus according to a sixth aspect of the invention, in the ultrasonic imaging apparatus according to the fourth or fifth aspect, the software determination means has a counter for counting the number of the software used therein, for each software of a different function.

As for the ultrasonic imaging apparatus according to a seventh aspect of the invention, in the ultrasonic imaging apparatus according to the sixth aspect, the software determination means increments the value of the counter by one, when the software is enabled by the determination.

As for the ultrasonic imaging apparatus according to an eighth aspect of the invention, in the ultrasonic imaging apparatus according to any one of the third to seventh aspects, the control unit has a display control section for causing the display unit to display list information of softwares whose number of uses is less than the maximum number.

In the eighth aspect, the available software is notified to an operator.

As for the ultrasonic imaging apparatus according to a ninth aspect of the invention, in the ultrasonic imaging apparatus according to the eighth aspect, the display control section causes the image display section of the display unit to display selection keys for selecting the softwares.

As for the ultrasonic imaging apparatus according to a tenth aspect of the invention, in the ultrasonic imaging apparatus according to the ninth aspect, the control unit has an operation control section for transmitting the selection information to the software determination means, based on selection operation of the selection keys.

In the tenth aspect, the operator easily starts the software.

As for the ultrasonic imaging apparatus according to an eleventh aspect of the invention, in the ultrasonic imaging apparatus according to the tenth aspect, the operation control section transmits end information to the software determination means, based on reselection operation of the selection keys.

As for the ultrasonic imaging apparatus according to a twelfth aspect of the invention, in the ultrasonic imaging apparatus according to any one of the second to eleventh aspects, the software determination means has a use period limitation means for limiting, when the software is enabled by the determination, the period for which the software is enabled in any one of the plurality of display units and the plurality of operation units.

In the twelfth aspect, the period of use of the software is limited, to avoid wasted use of the software, such as leaving the software unused.

As for the ultrasonic imaging apparatus according to a thirteenth aspect of the invention, in the ultrasonic imaging apparatus according to any one of the second to twelfth aspects, the software determination means has a loading means for loading, when the software is enabled by the determination, the software stored in the software management section in advance into the unit control means, so that the software is used in any one of the plurality of transmission/reception units, the plurality of display units, and the plurality of operation units.

In the thirteenth aspect, the software is loaded each time when the software is used.

As for the ultrasonic imaging apparatus according to a fourteenth aspect of the invention, in the ultrasonic imaging apparatus according to the thirteenth aspect, the loading means unloads the software loaded into the unit control section, based on the end information.

In the fourteenth aspect, the software is unloaded each time when the software is ended.

Further, an ultrasonic imaging system according to a fifteenth aspect of the invention, includes: a first ultrasonic imaging apparatus having a software offered by a source as well as a key code for enabling the software; a second ultrasonic imaging apparatus not having the software nor the key code; and a communication means for mutually communicating information between the first and second ultrasonic imaging apparatuses. The first ultrasonic imaging apparatus has a key code communication means for transmitting the key code with a limited period of use, to the second ultrasonic imaging apparatus through the communication means. The second ultrasonic imaging apparatus has a period-limited software using means for enabling the software by the key code, to use the software only for the period of use.

In the fifteenth aspect, the first ultrasonic imaging apparatus using the key code communication means, offers the key code with the limited period of use to the second ultrasonic imaging apparatus. The second ultrasonic imaging apparatus uses the period-limited software using means to run the software by the key code to use the software for a limited period of time.

As for the ultrasonic imaging system according to a sixteenth aspect of the invention, in the ultrasonic imaging system according to the fifteenth aspect, the first ultrasonic imaging apparatus has a communication information transmission means for transmitting communication information including destination information, to a server of the source, upon communication of the key code.

In the sixteenth aspect, the offering of the key code is acknowledged by the server of the source.

As for the ultrasonic imaging system according to a seventeenth aspect of the invention, in the ultrasonic imaging system according to the sixteenth aspect, the second ultrasonic imaging apparatus has a software reception means for receiving the software from the source or the first ultrasonic imaging apparatus, upon reception of the key code.

In the seventeenth aspect, the second ultrasonic imaging apparatus automatically receives the software.

By reducing the number of software licenses, it is possible to reduce the purchase cost and also reduce the software load in the control section, thereby making it suitable for the functionally-distributed ultrasonic imaging apparatus for performing centralized management in the control section.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the overall configuration of an ultrasonic imaging apparatus;

FIG. 2 is a bird's-eye view showing an example of ultrasonography rooms using the ultrasonic imaging apparatus shown in FIG. 1;

FIG. 3 is a block diagram showing a configuration of a software management section that may be used with the ultrasonic imaging apparatus shown in FIG. 1;

FIG. 4 is a block diagram showing a configuration of a display control section and an operation control section that may be used with the ultrasonic imaging apparatus shown in FIG. 1;

FIG. 5 is a flowchart showing the operation of the software management section shown in FIG. 3;

FIG. 6 is a diagram showing an example of the option software information that may be used with the ultrasonic imaging apparatus shown in FIG. 1;

FIG. 7 is a diagram showing an example of the list information that may be used with the ultrasonic imaging apparatus shown in FIG. 1;

FIG. 8 is a flowchart showing the operation of the software management section shown in FIG. 3; and

FIG. 9 is a block diagram showing a configuration of an exemplary ultrasonic imaging system.

### DETAILED DESCRIPTION OF THE INVENTION

Hereinafter, embodiments of the invention will be described with reference to the accompanying drawings. It is to be understood that the invention is not limited thereto.

### First Embodiment

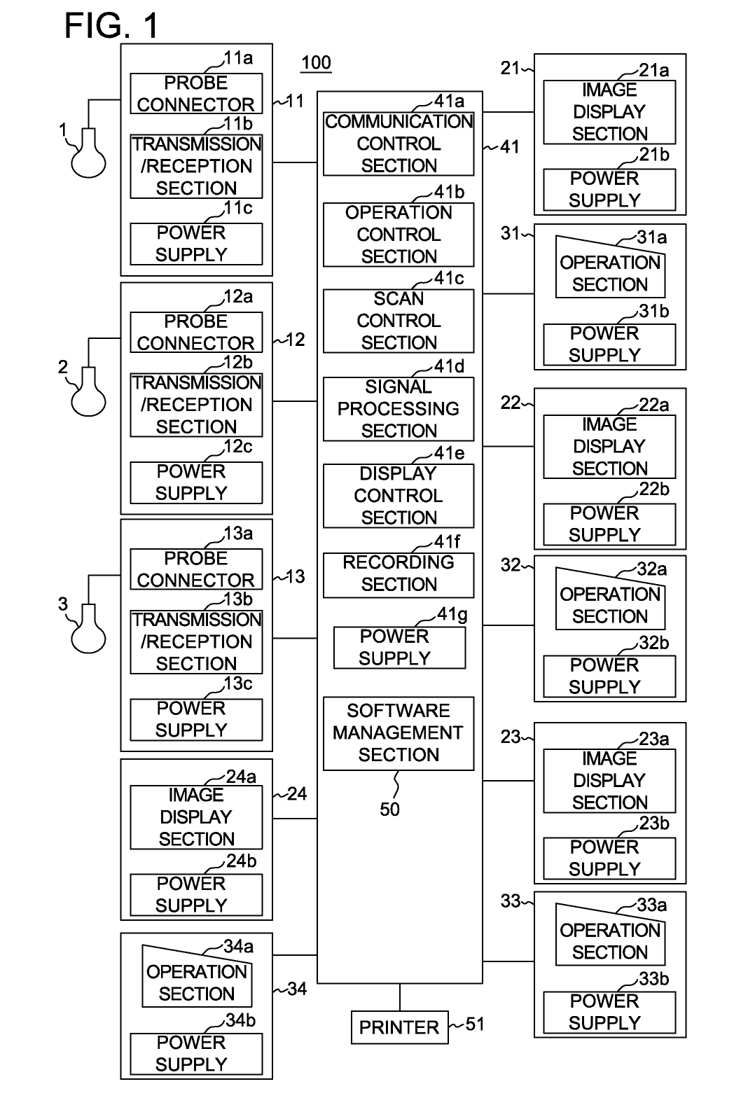

First, description will be given of the overall configuration of an ultrasonic imaging apparatus 100 according to a first embodiment. FIG. 1 is a diagram showing a configuration of the ultrasonic imaging apparatus 100 according to the first embodiment.

The ultrasonic imaging apparatus 100 includes: a plurality of ultrasonic probes 1, 2, 3; a plurality of transmission/reception units 11, 12, 13 to which the ultrasonic probes 1, 2, 3 are detachably connected, respectively; a plurality of display units 21, 22, 23, 24; a plurality of operation units 31, 32, 33, 34; a single control unit 41; and a printer 51 detachably connected to the control unit 41.

The transmission/reception unit 11 includes: a probe connector 11a for connecting the ultrasonic probe 1; a transmission/reception section 11b for driving the ultrasonic probe 1 to scan a subject by ultrasonic beams; and a power supply 11c. The transmission/reception unit 11 is detachably connected to the control unit 41.

The transmission/reception units 12, 13 have the same configuration as the transmission/reception unit 11.

The display unit 21 includes an image display section 21a and a power supply 21b. The display unit 21 is detachably connected to the control unit 41.

The display units 22, 23, 24 have the same configuration as the display unit 21.

The operation unit 31 includes an operation section 31a including a keyboard and a track ball, as well as a power supply 31b. The operation unit 31 is detachably connected to the control unit 41.

The operation units 32, 33, 34 have the same configuration as the operation unit 31.

The control unit 41 includes a unit control means including an operation control section 41b, a scan control section 41c, and a display control section 41e, together with a communication control section 41a, a signal processing section 41d, a recording section 41f, a power supply 41g, and a software management section 50.

In the control unit 41, the communication control section 41a performs wired communication with the transmission/reception units 11, 12, 13, the display units 21, 22, 23, 24, the operation units 31, 32, 33, 34, and the printer 51. The operation control section 41b performs appropriate processing according to the operation from the operation units 31, 32, 33, 34. The scan control section 41c scans the subject by ultrasonic beams, using the transmission/reception units 11, 12, 13 as well as the ultrasonic probes 1, 2, 3. The signal processing section 41d performs signal processing including generation of ultrasound images, based on the data obtained by scanning of ultrasonic beams. The display control section 41e displays the ultrasound images and the like on the corresponding display units 21, 22, 23, 24. The recording section 41f records the personal information, ultrasound images, and imaging parameters of the subject, the content of which is output to the printer 51.

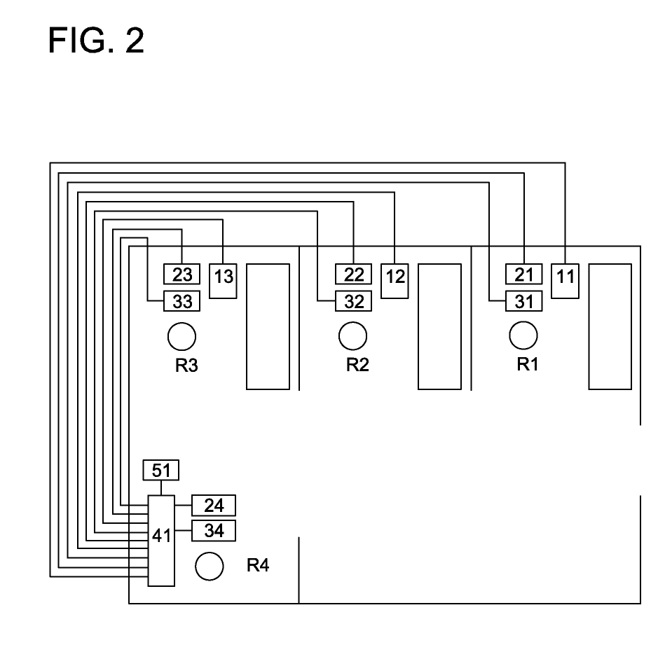

FIG. 2 is a bird's-eye view of ultrasonography rooms in which the transmission/reception units 11 to 13, the display units 21 to 24, the operation units 31 to 34, and the printer 51 are placed. The transmission/reception unit 11, the display unit 21, and the operation unit 31 are placed in a first ultrasonography room R1. The transmission/reception unit 12, the display unit 22, and the operation unit 32 are placed in a second ultrasonography room R2. The transmission/reception unit 13, the display unit 23, and the operation unit 33 are placed in a third ultrasonography room R3. The display unit 24, the operation unit 34, the control unit 41, and the printer 51 are placed in a control room R4. The transmission/reception units 11, 12, 13, the display units 21, 22, 23, and the operation units 31, 32, 33 are connected to the control unit 41 through the communication means.

A first sonographer of the first ultrasonography room R1 scans a first subject by means of the transmission/reception unit 11, the display unit 21, and the operation unit 31. The data obtained by scanning is transmitted from the transmission/reception unit 11 to the control unit 41. Based on the transmitted data, the control unit 41 generates an ultrasound image and the like, which is transmitted to the display unit 21. The transmitted ultrasound image and the like is displayed by the display unit 21.

A second sonographer of the second ultrasonography room R2 scans a second subject by means of the transmission/reception unit 12, the display unit 22, and the operation unit 32. The data obtained by scanning is transmitted from the transmission/reception unit 12 to the control unit 41. Based on the transmitted data, the control unit 41 generates an ultrasound image and the like, which is transmitted to the display unit 22. The transmitted ultrasound image and the like is displayed by the display unit 22.

A third sonographer of the third ultrasonography room R3 scans a third subject by means of the transmission/reception unit 13, the display unit 23, and the operation unit 33. The data obtained by scanning is transmitted from the transmission/reception unit 13 to the control unit 41. Based on the transmitted data, the control unit 41 generates an ultrasound image and the like, which is transmitted to the display unit 23. The transmitted ultrasound image and the like is displayed by the display unit 23.

When a doctor in the examination room R4 operates the control unit 34 to select the first ultrasonography room R1, an image including a part of the image displayed by the display unit 21, is displayed by the display unit 24. It is also possible to remotely control the transmission/reception unit 11, the display unit 21, and the operation unit 31 from the operation unit 34.

When the doctor in the examination room R4 operates the operation unit 34 to select the second ultrasonography room R2, an image including a part of the image displayed by the display unit 22, is displayed by the display unit 24. It is also possible to remotely control the transmission/reception unit 12, the display unit 22, and the operation unit 32 from the operation unit 34.

When the doctor in the examination room R4 operates the operation unit 34 to select the third ultrasonography room R3, an image including a part of the image displayed by the display unit 23, is displayed by the display unit 24. It is also possible to remotely control the transmission/reception unit 13, the display unit 23, and the operation unit 33 from the operation unit 34.

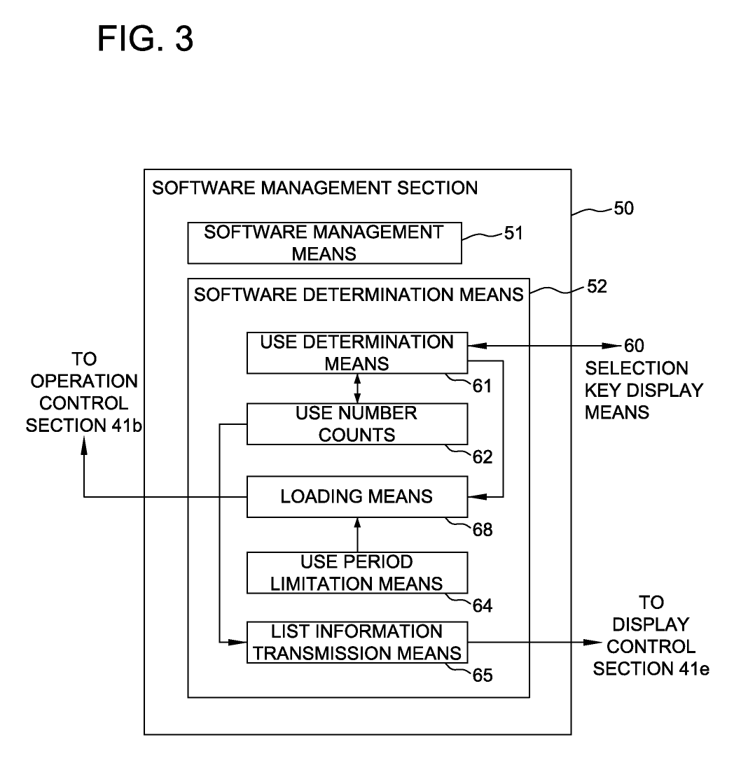

FIG. 3 is a block diagram showing a configuration of the software management section 50 in the control unit 41. The software management section 50 includes a software management means 51 and a software determination means 52. The software management means 51 manages the information relating to all the softwares used in the ultrasonic imaging apparatus 100. The software management means 51 also stores related information such as, for example, the type and version of the software used in the image display section 21a, and the date and time when the software was installed.

The software determination means 52 includes a use determination means 61, a use number counter 62, a loading means 68, a use period limitation means 64, and a list information transmission means 65.

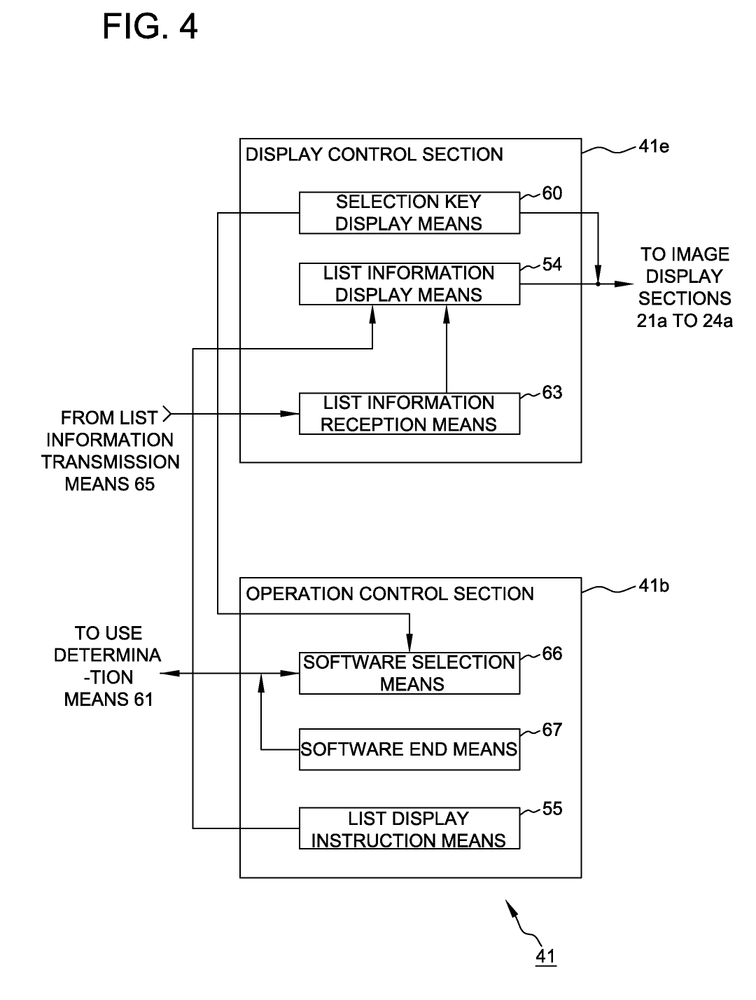

FIG. 4 is a block diagram showing a configuration of the display control section 41e and the operation control section 41b in the control unit 41. The display control section 41e includes a selection key display means 60, a list information display means 54, and a list information reception means 63. The operation control section 41b includes a software selection means 66, a software end means 67, and a list display instruction means 55. With respect to the functions of the use determination means 61, use number counter 62, loading means 68, use period limitation means 64, and list information transmission means 65 shown in FIG. 3, as well as the functions of the selection key display means 60, list information reception means 63, software selection means 66, software end means 67, and list display instruction means 55 shown in FIG. 4, will be described later in the description of the start and end operations of the ultrasonic imaging apparatus 100.

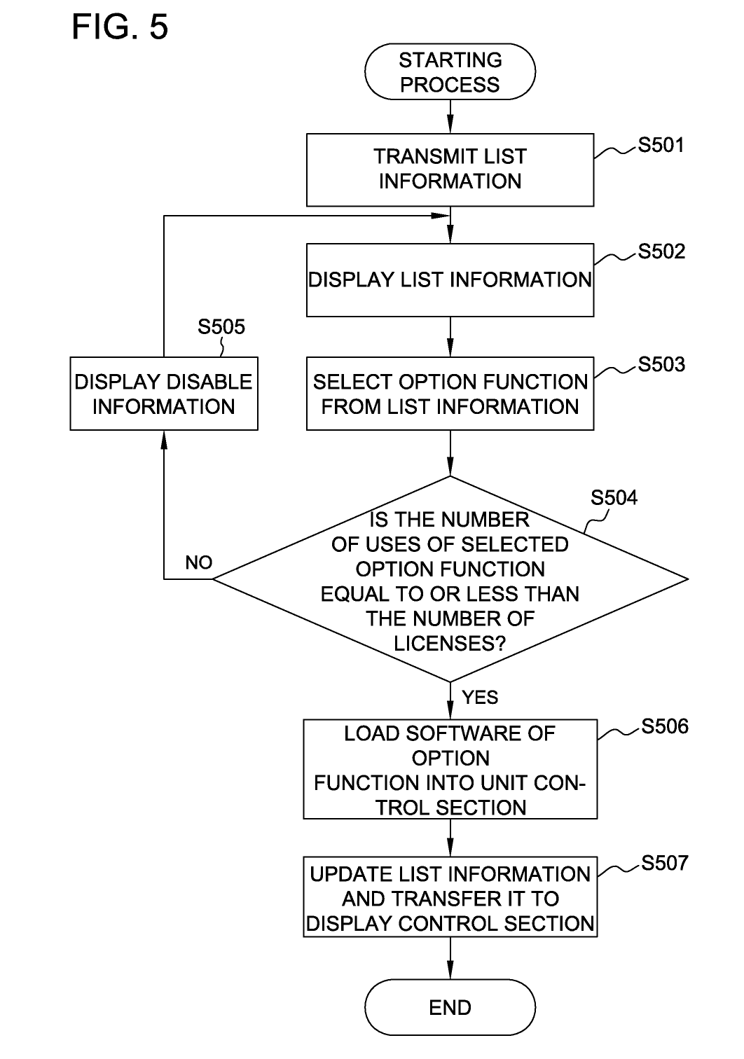

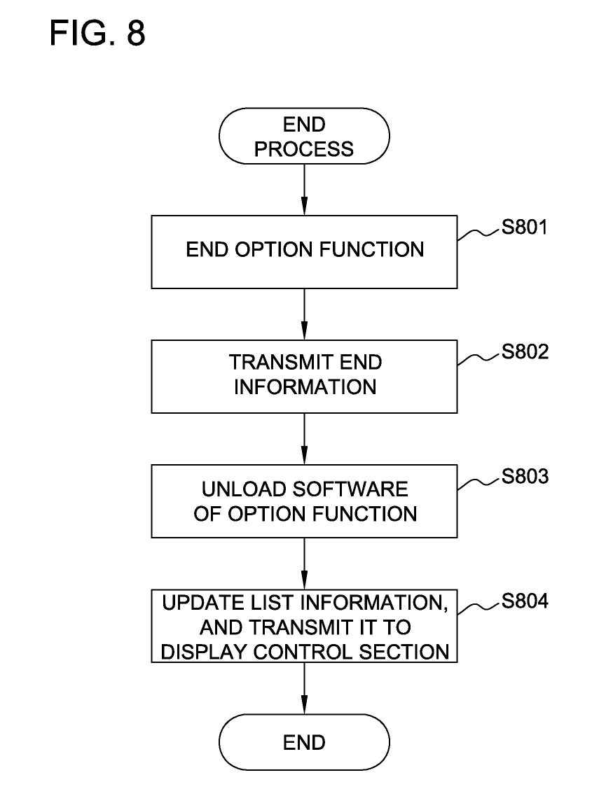

Next, the operation for starting or ending a software of an option function included in the ultrasonic imaging apparatus 100 according to the first embodiment, will be described with reference to the flowcharts of FIGS. 5 and 8. FIG. 5 is a flowchart showing the starting process for starting a software of an option function included in the ultrasonic imaging apparatus 100.

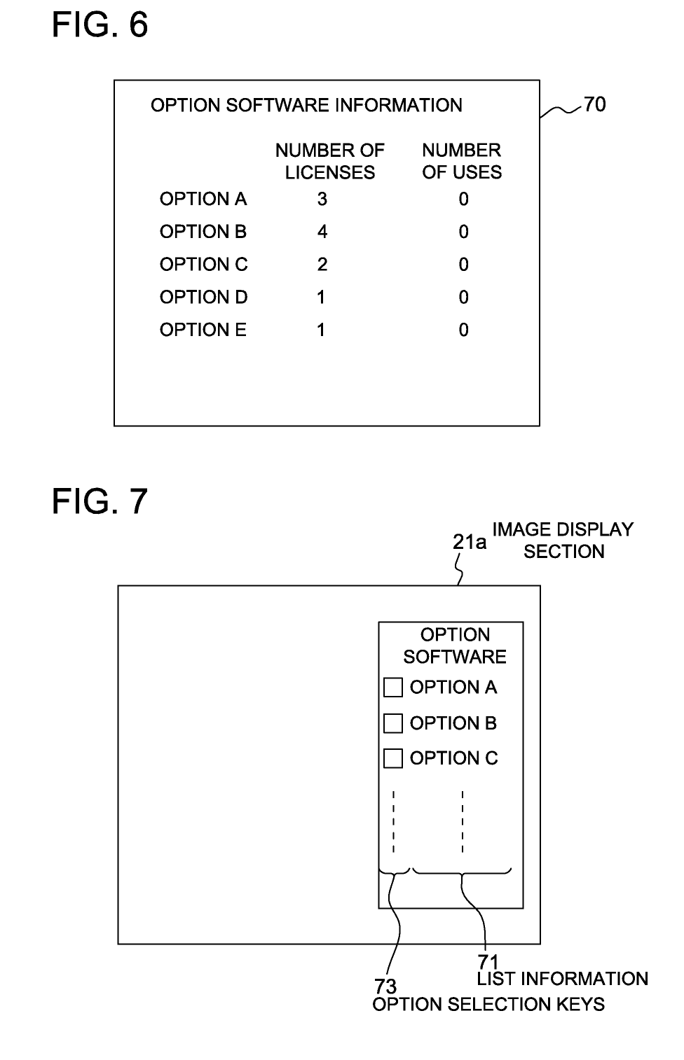

First, in the initial setting for starting the power supply, the list information transmission means 65 of the software management section 50 transmits list information 71 of available option functions, to the display control section 41e (Step S501). FIG. 6 is a diagram showing option software information 70 as the base of the list information 71 that is stored in the use determination means 61.

The option software information 70 includes a list of the names of the available option functions, each of which includes the number of licenses for use authorized by the source as well as the number of the corresponding option function being used. The option function is run by the software offered from the source with licensing terms and conditions. The number of licenses, which is the maximum number of the software to be used, is specified in the licensing terms and conditions. Incidentally, the term “use” herein includes an action such as copying, without involving execution of software.

In FIG. 6, it is shown that options A to E exist as available option functions in the option software information 70, and that the numbers of licenses of the options A to E are 3, 4, 2, 1, 1, respectively, with no option function used for transmission.

The list information transmission means 65 transmits the list information 71 containing only the names of the available option functions whose number of uses is less than the number of licenses and which are extracted from the option software information 70, to the display control section 41e. Incidentally, the list information reception means 63 of the display control section 41e receives the list information 71 transmitted from the list information transmission means 65, and inputs in the list information display means 54.

Then, it is assumed that the operator desires to use an option function of the control unit 41 in any one of the rooms R1 to R4 shown in FIG. 2, for example in the room R1. In this case, the operator displays the list information 71 stored in the list information display means 54 as well as the option selection keys 73, on the image display section 21a by inputting of the list display instruction means 55 including an input key or other input means in the operation section 31a (Step S502).

FIG. 7 is a diagram showing an example of the list information 71 and option selection keys 73 that are displayed on a display screen of the image display section 21a. In the display screen of the image display section 21a, the list information 71 of the option softwares as well as the option selection keys 73 are aligned together and displayed in one area on the right side of the screen. In this display screen, the selection keys are placed next to the names of the option functions of the displayed list information 71, in order to select each option by the corresponding selection key.

Then, returning to FIG. 5, the operator refers to the displayed list information 71, and selects the option function by the software selection means 66 (Step S503). In the software selection means 66, the operator moves a cursor to the option selection key 73 located on the left side of the name of the desired option function, for example, using a mouse of the operation section 31a, and selects the option selection key 73 by a key input. The software selection means 66 transmits the selection information of the selected option selection key 73 to the use determination means 61 of the software determination means 52.

Then, the software determination means 52 uses the use determination means 61 to determine whether the number of uses of the selected option function is less than the number of licenses, based on the selection information of the option function transmitted from the operation section 31a (Step S504). Here, the use number counter 62 stores the number of uses of each of the options A to E. The use determination means 61 refers to the use number counter 62, and determines whether the number of uses of the selected option function is less than the number of licenses. When the number of uses of the selected option function is not less than the number of licenses (negative in Step S504), the use determination means 61 transmits information that the option function is disabled, to the display control section 41e to display the information (Step S505), thereby causing the operator to acknowledge it.

On the other hand, when the number of uses of the selected option function is less than the number of licenses (positive in Step S504), the use determination means 61 uses the loading means 68 to load the software of this option function into the operation control section 41b that is the unit control means (Step S506). The loading means 68 loads the software of the specified operation function into the operation control section 41b based on a load instruction from the use determination means 61, so that the operator using the operation section 31a can use the operation function.

Then, the use determination means 61 increments by one the counter value of the use number counter 62 corresponding to the option function to be used. When there is an option function to be disabled due to the increment of the counter value, the list transmission means 65 updates the list information 71 by deleting the option function, and transmits the list information to the display control section 41e (Step S507), and then the starting process is ended.

FIG. 8 is a flowchart showing the end operation of the option function of the ultrasonic imaging apparatus 100 according to the first embodiment. First, using the operation control section 41b, the operator stops the operation performed by the software of the option function, and ends the use of the option function (Step S801). Here, the operator performs reselection of the option function keys 73 on the operation screen, which correspond to the respective option functions displayed on the operation screen of the image display section 21a. Alternatively, the operator performs selection of an end button, and the like, which is separately provided. The end information of the option function is transmitted to the use determination means 61 of the software determination means 52 (Step S802).

Then, by synchronizing the input of the end information, the use determination means 61 uses the loading means 68 to unload the software of the option function that was loaded into the operation control section 41b (Step S803). Further, the use determination means 61 decrements by one the counter value of the use number counter 62 corresponding to the used option function. When there is an option function to be enabled due to the decrement of the counter value, the list information transmission means 65 updates the list information 71 by adding the option function, and transmits the updated list information 71 to the display control section 41e (Step S804), and then the process is ended.

As described above, according to the first embodiment, the functionally-distributed ultrasonic imaging apparatus 100 is configured to have the software management section 50 in which the software determination means 52 is provided. The use determination means 61 of the software determination means 52 is provided so that the software of the option function whose frequency of use is low, is used in the operation control section 41b, the display control section 41e, and the signal processing section 41d, and the like, with less number of licenses according to the necessity. This makes it possible to increase the use efficiency of the software of the operation function, and to reduce the price of the software by reducing the number of licenses of the software without imposing a load on the operator. In addition, according to the first embodiment, it is possible to reduce the load applied to the control unit 41 in the operation of the software, preventing the functional reduction due to an excess load of the control unit 41, thereby increasing flexibility in the price or configuration of the ultrasonic imaging apparatus 100 at the time of purchase.

Further, in the first embodiment, the list information 71 only includes the names of the option functions available at the display time. However, it is also possible to display the list information 71 of all the softwares of the option functions provided in the control unit 41, including the numbers of licenses of the softwares as well as the numbers of uses at the display time. In this way, the operator can accurately know the use state of the software of the corresponding option function in the ultrasonic imaging apparatus 100, and can use the software more effectively.

Further, in the first embodiment, the started software of the option function is unloaded from the operation control section 41b at the end of use. However, it is also possible to limit the period of use by the use period limitation means 64 at the time when the software is loaded into the operation control section 41b. The use period limitation means 64 provides a timer to the software to be loaded. The operation control section 41b automatically stops the use of the software when a predetermined period of time has passed from the load of the software, for example, when one day or one week or more has passed. Then, the operation control section 41b transmits the end information to the use determination means 61 through a signal line not shown. The use determination means 61 performs the same end process as described above, based on the end information. In this way, it is possible to reduce wasteful time in the use of the option function that occurs due to the operator leaving the software in a used state or other causes.

Further, in the first embodiment, the description has been given of the software of the option function used in the operation section 31a. However, the operation is exactly the same for the softwares of the operation functions used in the other operation sections 32a to 34a, the transmission/reception sections 11b to 13b, and the image display sections 21a to 24a.

Further, in the first embodiment, the description has been given of the case of using the software of the option function. However, the invention is not limited to the software of the option function, and any software can be used as long as the frequency of use is low.

Further, in the first embodiment, the communication means 41a performs wired communication. However, wireless communication can also be used exactly in the same manner. In the case of using wireless communication, the communication control section 41a and the respective units each have a wireless transceiver.

### Second Embodiment

As described above, according to the first embodiment, the functionally-distributed ultrasonic imaging apparatus 100 is configured such that the software of the option function is effectively used by reducing the number of licenses for use of the software of the option function, and by loading the software into the operation control section, the display control section, and the like, according to the necessity. However, in a plurality of integrated ultrasonic imaging apparatuses connected to each other via a communication network, it is also possible to effectively use the software of the option function whose frequency of use is low, by moving the software between the apparatuses according to the necessity. Thus, a second embodiment shows the case where one software of option function is effectively used between a plurality of ultrasonic imaging apparatuses connected via a network.

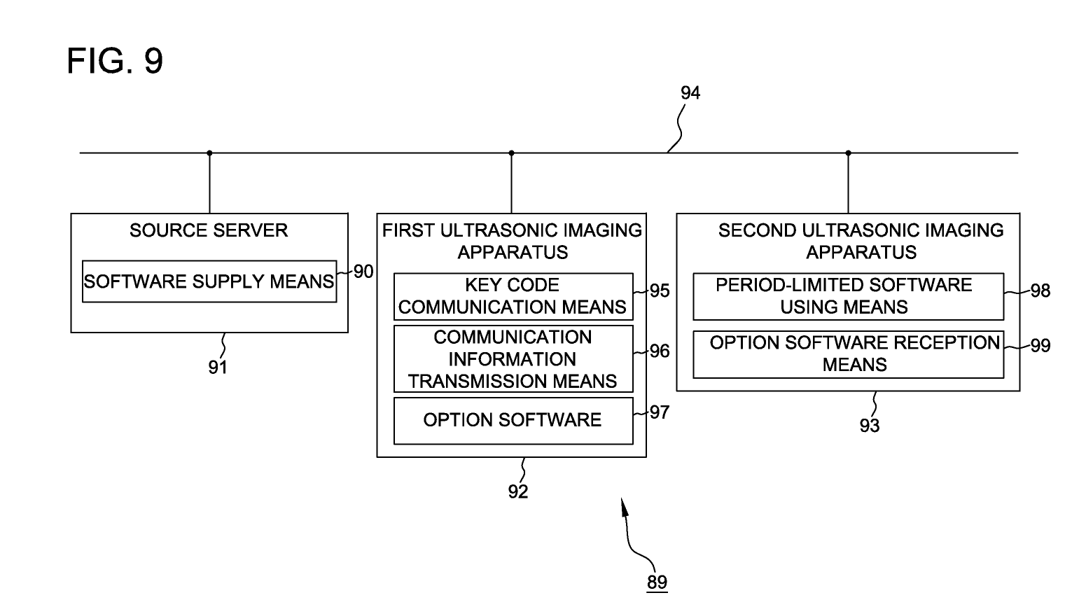

FIG. 9 is a block diagram showing a configuration of an ultrasonic imaging system 89 according to the second embodiment. The ultrasonic imaging system 89 includes a source server 91, a first ultrasonic apparatus 92, a second ultrasonic apparatus 93, and a communication means 94.

The communication means 94 performs transmission/reception of information with the source server 91, the first ultrasonic imaging apparatus 92, and the second ultrasonic imaging apparatus 93 through a wired cable.

The source server 91 is a server located in a source, such as in the office of a manufacturer for manufacturing the software of option function, or in the office of a distributor for selling the software of option function. The source server 91 includes a software supply means 90. The software supply means 90 installs the option software to an ultrasonic imaging apparatus connected to a communication means 94, namely, such as the first ultrasonic imaging apparatus 92 or the second ultrasonic imaging apparatus 93, in response to requests from administrators of the ultrasonic imaging apparatuses.

Further, the source server 91 also manages key codes for enabling the option software, which will be described later. For example, the source server 91 manages the key codes transmitted from the first and second ultrasonic imaging apparatuses, to manage which of the ultrasonic imaging apparatuses is using the option software.

The first ultrasonic imaging apparatus 92 is located in a hospital, and includes an option software 97, a key code communication means 95, and a communication information transmission means 96. The option software 97 is a software of option function that a first administrator of the first ultrasonic imaging apparatus 92 has purchased.

The option software 97 is a software whose frequency of use is low, which is enabled by starting to install executable software by an input of a key code supplied from the manufacture or the distributor. Incidentally, the key code is prevented from being copied by encryption or other obfuscation techniques.

The key code communication means 95 transfers the key code of the option software 97 to another ultrasonic imaging apparatus connected to the communication means 94, for example, to the second ultrasonic imaging apparatus 93, in response to an instruction from the first administrator of the option software 9.

Upon transfer of the key code to another administrator, the communication information transmission means 96 transfers the information of the key code transfer as well as the information of the key code destination, to the source server 91.

The second ultrasonic imaging apparatus 93 is of a second administrator in a hospital other than the hospital in which the first ultrasonic imaging apparatus 92 exists. The second ultrasonic imaging apparatus 93 includes an option software reception means 99 and a period-limited software using means 98. Upon receiving the key code, the option software reception means 99 reads the corresponding option software from the first ultrasonic imaging apparatus 92 or the source server 91. The period-limited software using means 98 enables the input key code and option software by converting to an executable format with a limited period. Further, when the use period is expired, the period-limited software using means 98 returns the option software to the source server 91 or the first ultrasonic imaging apparatus 92 that is the source of the option software, and also returns the corresponding key code to the first ultrasonic imaging apparatus 92.

Next, description will be given of the operation of the ultrasonic imaging system 89 according to the second embodiment. First, the first administrator purchases the option software 97 from the source, and installs to the first ultrasonic imaging apparatus 92 to use the option software 97.

It is assumed that, based on the experience of using the option software 97, the first administrator wants the second administrator having the similar second ultrasonic imaging apparatus 93 to use the option software while not using the option software 97. In this case, the first administrator transmits the key code of the option software 97 from the ultrasonic imaging apparatus 92 to the second ultrasonic imaging apparatus 93.

The second ultrasonic imaging apparatus 93 reads the option software 97 from the source server 91 or the first ultrasonic imaging apparatus 92 by the option software reception means 99 upon input of the key code. Then, the second ultrasonic imaging apparatus 93 enables the option software 97 with a limited period by the period-limited software using means 98.

The second administrator actually uses the operational software 97 for a predetermined period using the second ultrasonic imaging apparatus 93.

After the predetermined period has expired, the second ultrasonic imaging apparatus 93 deletes the option software 97 or returns it to the source, and also returns the key code to the first ultrasonic imaging apparatus 92, and then the process is ended. Incidentally, the first administrator is not allowed to use the option software 97 without the key code during this period.

As described above, in the second embodiment, when the first administrator does not use the option software 97, the option software 97 is offered to the second administrator for a predetermined period and is enabled. The second administrator can actually use the option software 97 by the second ultrasonic imaging apparatus 93 and confirm its effectiveness. In this way, it is possible to achieve rapid promotion of the product between users using the same apparatus as the first ultrasonic imaging apparatus 92.

Although the second embodiment has shown the case where the communication means 94 uses the wired cable, it is also possible to use wireless apparatuses or a combination of wireless apparatuses and wired cable in exactly the same manner. In this case, when wireless communication is performed between wireless apparatuses, for example, the first ultrasonic imaging apparatus 92 and the second ultrasonic imaging apparatus 93, the first ultrasonic imaging apparatus 92 and the second ultrasonic imaging apparatus 93 each have a wireless transmission/reception section.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus comprising:
a plurality of transmission/reception units each comprising a probe connector, a transmission/reception section, and a power supply;
a plurality of display units each comprising an image display section and a power supply;
a plurality of operation units each comprising an operation section and a power supply;
a single control unit; and
a communication means configured to communicatively couple said control unit to said plurality of transmission/reception units, said plurality of display units, and said plurality of operation units, wherein said control unit comprises:
a unit control means configured to individually control said plurality of transmission/reception units, said plurality of display units, and said plurality of operation units, all of which are connected to said control unit; and
a software management section configured to selectively enable and disable software for respectively controlling said plurality of display units and said plurality of operation units.

2. The ultrasonic imaging apparatus according to claim 1, wherein said software management section comprises a software determination means configured to selectively enable and disable the software based on selection information for selecting the software from at least one of said plurality of operation units through said unit control means.

3. The ultrasonic imaging apparatus according to claim 2, wherein the software is an option software which is not included as standard equipment in said ultrasonic imaging apparatus.

4. The ultrasonic imaging apparatus according to claim 2, wherein the software is offered with licensing terms and conditions in which a maximum number of the software to be used is specified, and wherein said software determination means is configured to determine whether a number of uses of the software in said unit control means is equal to or less than the maximum number.

5. The ultrasonic imaging apparatus according to claim 3, wherein the software is offered with licensing terms and conditions in which a maximum number of the software to be used is specified, and wherein said software determination means is configured to determine whether a number of uses of the software in said unit control means is equal to or less than the maximum number.

6. The ultrasonic imaging apparatus according to claim 4, wherein the use includes copying for only making copies, without involving execution of the software.

7. The ultrasonic imaging apparatus according to claim 4, wherein said software determination means comprises a counter configured to count the number of of uses of the software for each software of a different function.

8. The ultrasonic imaging apparatus according to claim 6, wherein said software determination means comprises a counter configured to count the number of of uses of the software for each software of a different function.

9. The ultrasonic imaging apparatus according to claim 7, wherein said software determination means configured to increment the value of said counter by one when the software is enabled by said software determination means.

10. The ultrasonic imaging apparatus according to claim 3, wherein said control unit comprises a display control section configured to cause at least one of said plurality of display units to display list information of software whose number of uses is less than a maximum number.

11. The ultrasonic imaging apparatus according to claim 4, wherein said control unit comprises a display control section configured to cause at least one of said plurality of display units to display list information of software whose number of uses is less than a maximum number.

12. The ultrasonic imaging apparatus according to claim 10, wherein said display control section is configured to cause an image display section of at least one of said plurality of display units to display selection keys for selecting the software.

13. The ultrasonic imaging apparatus according to claim 12, wherein said control unit comprises an operation control section configured to transmit selection information to said software determination means, based on selection operation of the selection keys.

14. The ultrasonic imaging apparatus according to claim 13, wherein said operation control section is configured to transmit end information to said software determination means, based on a reselection operation of the selection keys.

15. The ultrasonic imaging apparatus according to claim 2, wherein said software determination means comprises a use period limitation means configured to limit, when the software is enabled by said software determination means, a period in which the software is enabled in any one of said plurality of transmission/reception units, said plurality of display units, and said plurality of operation units.

16. The ultrasonic imaging apparatus according to claim 2, wherein said software determination means comprises a loading means configured to load, when the software is enabled by said software determination means, the software stored in said software management section in advance into said unit control means, such that the software is used in any one of said plurality of transmission/reception units, said plurality of display units, and said plurality of operation units.

17. The ultrasonic imaging apparatus according to claim 16, wherein said loading means is configured to unload software loaded into said unit control means, based on the end information.

18. An ultrasonic imaging system comprising:
a first ultrasonic imaging apparatus comprising a software offered by a source, and a key code for enabling the software;
a second ultrasonic imaging apparatus that does not store software or the key code; and
a communication means configured to mutually communicate information between said first and second ultrasonic imaging apparatuses,
wherein said first ultrasonic imaging apparatus comprises a key code communication means configured to transmit the key code with a limited period of use, to said second ultrasonic imaging apparatus via said communication means, and
wherein said second ultrasonic imaging apparatus comprises a period-limited software using means configured to enable the software by the key code, to use the software only for the period of use.

19. The ultrasonic imaging system according to claim 18, wherein, upon communication of the key code, said first ultrasonic imaging apparatus comprises a communication information transmission means configured to transmit communication information including destination information to a server of the source.

20. The ultrasonic imaging system according to claim 19, wherein said second ultrasonic imaging apparatus comprises a software reception means configured to receive the software from one of the source and said first ultrasonic imaging apparatus, upon reception of the key code.
